(19) United States
(12) Patent Application Publication
(10) Pub. No.: US 2004/0254251 A1
(43) Pub. Date: Dec. 16, 2004

(54) Memantine oral dosage forms

(75) Inventors: Bruce A. Firestone, Irvine, CA (US); J. Jacob Vander Zanden, Dana Point, CA (US); Rodney J. Terwilliger, Dana Point, CA (US); Janet K. Cheetham, Laguna Niguel, CA (US); Richard Kurjan, Huntington Beach, CA (US); Teresa H. Kuan, Placentia, CA (US); Chin-Ming Chang, Tustin, CA (US); J. Abraham M. Espiritu, Oceanside, CA (US)

Correspondence Address: Brent A. Johnson, Allergan, Inc., 2525 Dupont Drive, Irvine, CA, 92612, US

(73) Assignee: ALLERGAN, INC., Irvine, CA

(21) Appl. No.: 10/869,169

(22) Filed: Jun. 15, 2004

Related U.S. Application Data
(60) Provisional application No. 60/478,979, filed on Jun. 16, 2003.

(51) Int. Cl. A61K 31/13

(52) U.S. Cl. 514/662

(57) ABSTRACT

This invention relates to an oral dosage form containing between 1 mg and 100 mg of memantine, wherein said dosage form does not contain 10 mg of memantine or 20 mg of memantine, and wherein said dosage form is not prepared by the patient or a person administering the drug to the patient who divides the dosage form containing a larger dose of memantine. Other aspects of this invention relate to pharmaceutical products comprising said dosage forms and methods of administering memantine and treating disease with said dosage form.

### CROSS REFERENCE TO RELATED APPLICATIONS

This application claims priority under 35 U.S.C. § 119(e)(1) to provisional application No. 60/478,979 which was filed on Jun. 16, 2003.

### FIELD OF THE INVENTION

This invention relates to the pharmaceutical dosage forms. More specifically, this invention relates to oral dosage forms of memantine.

### DESCRIPTION OF THE RELATED ART

Mentamine is a drug that is believed to be useful to prevent nerve cell loss in glaucoma patients. It is also useful in the treatment of Alzheimer's Dementia. To avoid adverse effects associated with the drug, memantine is initially administered to the patient at a dose of 5 mg per day, which is gradually increased to a maintenance dose of either 10 mg or 20 mg per day. The dosage is increased by 5 mg biweekly until the maintenance dose is reached.

Currently two memantine products are marketed in Europe for the treatment of Alzheimer's Dementia. Both products, Axura® (Merz) and Ebixa® (Lundbeck) are marketed in packages of 10 mg tablets. The currently available products require the patient to track the dose that they are required to take according to how long they have been taking the drug, and to take ½ tablet, 1 tablet, 1½ tablets, or 2 tablets accordingly. Because the typical patient requiring this medication is using it to slow the progression of blindness or dementia, patients taking memantine are particularly susceptible to incorrect dosing with this type of graduated dose schedule. In particular, it is likely that the 5 mg and 15 mg doses will be especially problematic for patients because they are required to both choose the correct tablets and divide the 10 mg tablet in half. Furthermore, the patients must keep track of the half tablet left over after dividing the tablet for use in the next day. Finally, dividing a tablet introduces the possibility that the dose will not be consistent.

### SUMMARY OF THE INVENTION

### BRIEF DESCRIPTION OF THE INVENTION

We have found that dosage forms of memantine that contain doses of memantine that are not 10 mg or 20 mg are useful in helping to overcome the difficulties described above. These dosage forms contain between 1 mg and 100 mg of memantine. Unlike other dosage forms of memantine containing a dose that is not 10 mg or 20 mg, these dosage forms are not prepared by the patient or the person administering the medication to the patient who divides a larger dose. In other words, the patient or the person administering the medication does not have to divide the dosage form to obtain the appropriate dose. While not intending to be limiting in any way, this invention for example, provides for 5 mg and 15 mg tablets of memantine that are available to the patient or the person administering the drug to the patient in those forms, meaning that the patient does not have to divide a 10 mg tablet to obtain a 5 mg dose or take 1½ 10 mg tablets to obtain a 15 mg dose. Another significant contribution this invention makes to the art is that it allows a maintenance dose to be administered that is not 10 or 20 mg. For example, if a person needs more than 10 mg daily of memantine, but a 20 mg daily dose is undesirable, the person could receive a 15 mg maintenance dose without having difficulties with misdosing.

### BRIEF DESCRIPTION Of THE DRAWING FIGURES

FIG. 1 shows the manufacturing process diagram for the 175 Kg process for Memantine HCl tablets.

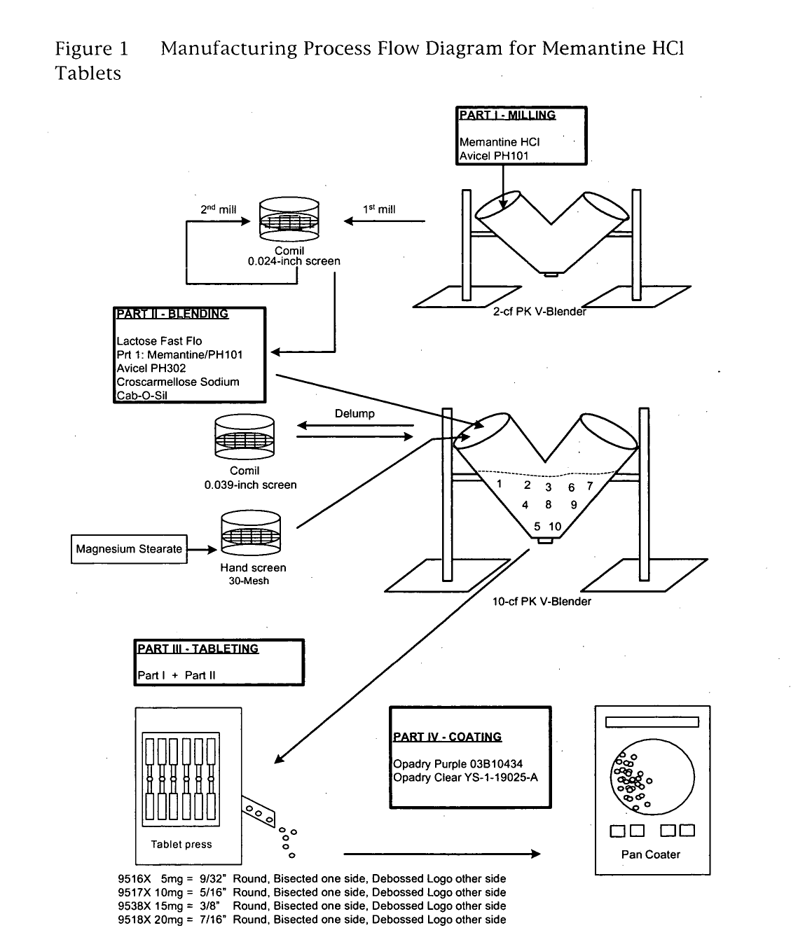

FIG. 2 shows the manufacturing process flow chart for the 175 Kg process for Memantine HCl tablets.

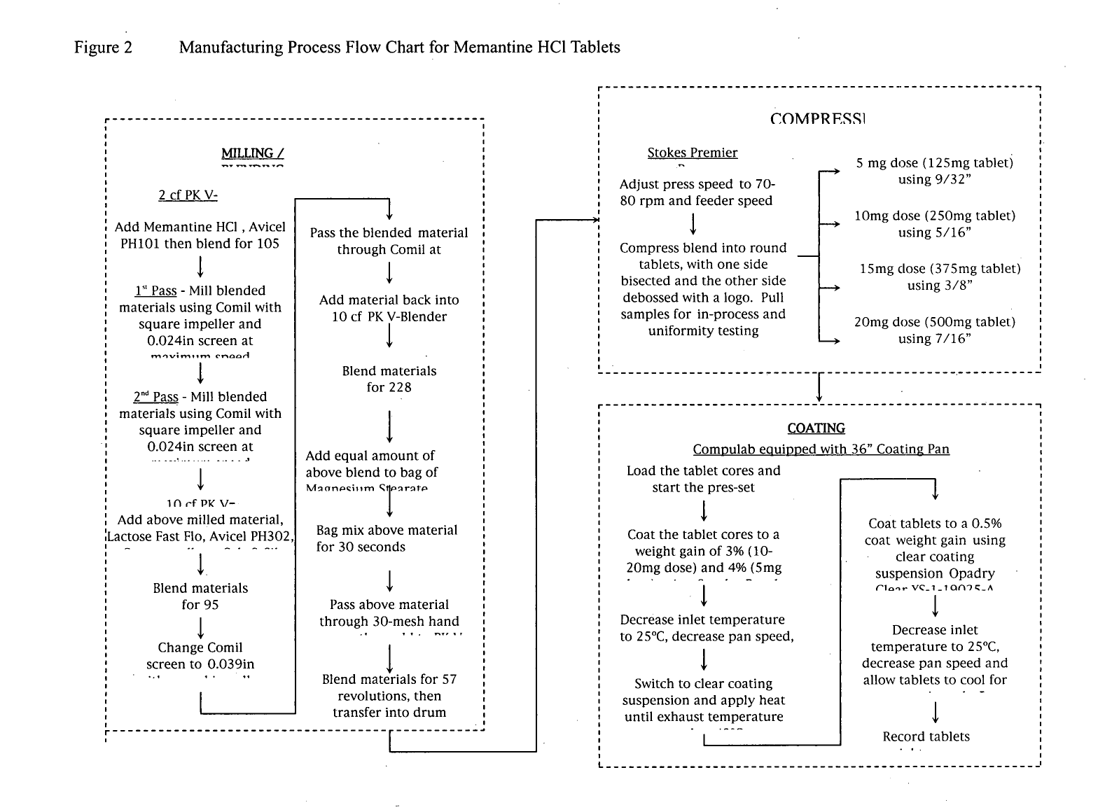

### DETAILED DESCRIPTION OF THE INVENTION

One aspect of this invention relates to an oral dosage form containing between 1 mg and 100 mg of memantine, wherein said dosage form does not contain 10 mg of memantine, and wherein said dosage form is not prepared by the patient or a person administering the drug to the patient who divides the dosage form containing a larger dose of memantine. Preferably, said oral dosage form contains 5 mg,15 mg or 20 mg of memantine, where the particular dose of memantine used in relation to this invention is determined by the required dose of the patient according to the dosage schedule described above.

In another aspect of this invention the oral dosage form is a solid dosage form, preferably a tablet.

Another embodiment of this invention relates to a method of administering memantine to a patient in amount that is not 10 mg, comprising administering to the patient an oral dosage form of memantine, wherein said dosage form is not prepared by the patient or a person administering the drug to the patient who divides the dosage form containing a larger dose of memantine.

Another aspect of this invention relates to a packaged pharmaceutical product comprising individual oral dosage forms of memantine which contain 5 mg, 15 mg, or 20 mg of memantine.

Preferably the packaged pharmaceutical product comprises individual oral dosage forms containing 5 mg of memantine, 10 mg of memantine, 15 mg of memantine, and 20 mg of memantine. In another preferred embodiment of this invention the packaged pharmaceutical product comprises individual oral dosage forms containing 5 mg of memantine and 10 mg of memantine.

In certain cases, the initial administration of 5 mg of memantine, or the increase in the dosage by 5 mg increments, may be more than can be tolerated by the patient. Another aspect of this invention relates to a method of treating a patient with memantine, comprising

a. administering a gradually increasing dose of memantine to said patient until the maintenance dose is reached, and

b. continuing to administer said maintenance dose on a regular basis as long as memantine is needed,

wherein the maintenance dose is reached in a sufficiently long period of time to significantly reduce adverse events, wherein the increment in which the dose of memantine is increased in successive dosages is less than 5 mg of memantine. The term adverse event refers to any undesirable side effect or toxic effect associated with memantine. In relation to embodiments of this type, it is preferable that the dose of memantine is increase by an increment of about 0.25 to about 0.5 mg each day until the maintenance dose is reached.

The best mode of making and using the present invention are described in the following examples. These examples are given only to provide direction and guidance in how to make and use the invention, and are not intended to limit the scope of the invention in any way.

### EXAMPLE 1

Unless otherwise indicated, all steps in this procedure are carried out at room temperature. Table 1, below, shows the amounts of each ingredient used in this procedure. In a 2 cubic foot PK V-Blender, memantine HCl (Conti BPC N.V., Landen, Belgium) and microcrystalline Cellulose (Avicel PH101, FMC Corporation, Philadelphia, Pa.) are combined and mixed for 105 revolutions. The mixture is then milled with a Quadro Comil using a 0.024-inch (0.6-mm) screen and a square impeller. The milling step is repeated for a second time. The milled mixture is then filled into polyethylene lined drum.

In a 10 cubic foot PK V-blender, the milled memantine HCl/microcrystalline cellulose mixture is combined with the Lactose, microcrystalline cellulose (Avicel PH302, FMC Corporation, Philadelphia, Pa.), Crosscarmellose sodium (FMC Biopolymer, Philadelphia, Pa.), and colloidal silicon dioxide (Cab-O-Sil, Cabot Corporation, Tuscola, Ill.). The mixture is mixed for 95 revolutions and then passed through the 0.039-inch (1-mm) screen using the Quadro Comil with round impeller. The mixture is placed back into the V-blender and mixed for 228 revolutions. The Magnesium Stearate is manually passed through a 30-mesh (0.6-mm) screen and added into the V-blender. The mixture is mixed for final 57 revolutions. For the validation batches, blend samples are taken at 10 different locations (See FIG. 5.2.1) using stainless steel side sample thief with 0.5-cc insert. The blend samples are tested for blend uniformity prior to the compression of tablets. The blend is charged into polyethylene lined drums.

The tablet press equipment is set up with appropriate punch tooling to produce each of the four dose strengths. The upper punches are embossed with the appropriate logo, and the lower punches are bisected (tablet scoring).

The Memantine HCl blend is manually scooped from the polyethylene lined drum into the press hopper. The press is then set to the appropriate compression parameters to produce the required in-process tablet specifications. The blend is compressed at the appropriate compression rate to produce the required tablets lot size per dose strength. During the compression, the tablet weight and hardness are monitored periodically as the tablets are collected in a polyethylene-lined drum. For the validation batches, tablet samples are collected at minimum frequency of beginning, middle and end of the compression run. These samples are tested for content uniformity prior to the film-coating step.

The coating procedure is carried out with the ingredients shown in the amounts listed in Table 2. The coating equipment is set up with a 36-inch pan and three spray guns. The first coating suspension is prepared with Colorcon's (West Point, Pa.) Opadry Purple 03B10434 at 12% (w/w). This material contains titanium dioxide, FD&C Blue #2 and Red #40 (purple colorant), hydroxypropyl methylcellulose (HPMC; polymer carrier) and polyethylene glycol (PEG) 400 (plasticizer). The second suspension is prepared with Opadry Clear YS-1-19025-A material at 5% (w/w). This clear coating material contains HPMC and PEG.

After the coating pan is charged with 50 kg of appropriate tablet dose strength, the coating equipment is set up with the appropriate parameters detailed in the coating batch records. The purple coating solution is applied onto the tablets at the appropriate spray rate until the required amount is achieved. The film-coat is applied at 4% of core weight for 5 mg dose and at 3% for the 10-20 mg dose tablets. Using the same coating parameters, a final coat with the clear coating solution at 0.5% of each core weight is applied. The tablets are allowed to dry in the coating pan for a short period prior to transfer into a polyethylene-lined drum.

TABLE 1
Ingredient Quantities for 175 Kg Lots of Memantine HCl
Blend

| | | | | Quantity for |
|---|---|---|---|---|
| | | | Concentration | 175 kg Lot |
| Part | Description | Ingredient | (% w/w) | (kg) |
| Part I | Milling | Memantine HCl | 4.00 | 7.00 |
| | | Avicel PH101 | 4.00 | 7.00 |
| Part II | Blending | Part I | — | — |
| | | Lactose Fast Flo | 69.61 | 121.82 |
| | | Avicel PH302 | 16.84 | 29.47 |
| | | Croscarmellose | 5.00 | 8.75 |
| | | Sodium | | |
| | | Cab-O-Sil | 0.25 | 0.44 |
| | | Magnesium | 0.30 | 0.52 |
| | | Stearate | | |

TABLE 2
Ingredient Quantities for Film Coating of 50 Kg of
Memantine HCl Tablets

| | Required Amounts (kg) | |
|---|---|---|
| | for 50 Kg of Tablets for Coating | |
| Film Coating Ingredient | 5 mg dose | 10, 15, 20 mg doses |
| Purple Coating | | |
| Opadry Purple 03B10434 | 2.00 | 1.50 |
| Water | 14.67 | 11.00 |
| Glaze Coating | | |
| Opadry Clear YS-1-19025-A | 0.25 | 0.25 |
| Water | 4.75 | 4.75 |

### EXAMPLE 2

To a patient suffering from glaucoma, is administered a tablet comprising 5 mg of memantine, prepared according to example 1, daily for two weeks. No misdosing occurs. After two weeks, a tablet comprising 10 mg of memantine is administered daily for as long as the drug is needed.

### EXAMPLE 3

To a patient suffering from glaucoma, is administered a tablet comprising 5 mg of memantine, prepared according to example 1, daily for two weeks. No misdosing occurs. After two weeks, a tablet comprising 10 mg of memantine is administered daily for two weeks. At the beginning of the fourth week of the treatment, a tablet comprising 15 mg of memantine, prepared according to Example 1, is administered daily for two weeks. No misdosing occurs. At the beginning of the sixth week of treatment, a tablet comprising 20 mg of memantine is administered daily for as long as the drug is needed.

### EXAMPLE 4

To a patient suffering from glaucoma, is administered a tablet comprising 2 mg of memantine is administered for one week. The patient then receives a tablet comprising a 4 mg dose for one week. The dose is increased by 2 mg each week until a 10 mg dose is reached at the beginning of the fifth week. During the first four weeks of administration of the drug, improved tolerance of the drug is observed. The tablet comprising 10 mg of memantine is administered daily for as long as the drug is needed.

### EXAMPLE 5

To a patient suffering from glaucoma, is administered a tablet comprising 2 mg of memantine is administered for one week. The patient then receives a tablet comprising a 4 mg dose for one week. The dose is increased by 2 mg each week until a 20 mg dose is reached at the beginning of the tenth week. During the first nine weeks of administration of the drug, improved tolerance of the drug is observed. The tablet comprising 20 mg of memantine is administered daily for as long as the drug is needed.

### EXAMPLE 6

To a patient suffering from glaucoma, is administered a tablet comprising 5 mg of memantine, prepared according to example 1, daily for two weeks. No misdosing occurs. After two weeks, a tablet comprising 10 mg of memantine is administered daily for two weeks. At the beginning of the fourth week of the treatment, a tablet comprising 15 mg of memantine, prepared according to Example 1, is administered daily for as long as the drug is needed. No misdosing occurs.

What is claimed is:

1. An oral dosage form containing between 1 mg and 100 mg of memantine, wherein said dosage form does not contain 10 mg of memantine, and wherein said dosage form is not prepared by the patient or a person administering the drug to the patient who divides the dosage form containing a larger dose of memantine.

2. The oral dosage form of claim 1 which contains 5 mg or 15 mg of memantine.

3. The oral dosage form of claim 2 which is a solid dosage form.

4. The oral dosage form of claim 3 which is a tablet.

5. The oral dosage form of claim 3 which contains 5 mg of memantine.

6. The oral dosage form of claim 3 which contains 15 mg of memantine.

7. The oral dosage form of claim 3 which contains 20 mg of memantine.

8. A method of administering memantine to a patient in amount that is not about 10 mg, comprising administering to the patient an oral dosage form of memantine, wherein said dosage form is not prepared by the patient or a person administering the drug to the patient who divides the dosage form containing a larger dose of memantine.

9. The method of claim 8 wherein said dosage form contains 5 mg or 15 mg of memantine.

10. The method of claim 9 wherein said dosage form is a solid dosage form,

11. The method of claim 10 wherein said dosage form is a tablet.

12. The method of claim 10 wherein said tablet contains 5 mg of memantine.

13. The method of claim 10 wherein said tablet contains 15 mg of memantine.

14. A packaged pharmaceutical product comprising individual oral dosage forms of memantine which contain 5 mg, 15 mg, or 20 mg of memantine.

15. The packaged pharmaceutical product of claim 14 which comprises individual oral dosage forms containing 5 mg of memantine, 10 mg of memantine, 15 mg of memantine, and 20 mg of memantine.

16. The packaged pharmaceutical product of claim 14 which comprises individual oral dosage forms containing 5 mg of memantine and 10 mg of memantine.

17. A method of treating a patient with memantine, comprising
a. administering a gradually increasing dose of memantine to said patient until the maintenance dose is reached, and
b. continuing to administer said maintenance dose on a regular basis as long as memantine is needed,
wherein the maintenance dose is reached in a sufficiently long period of time to significantly reduce adverse events, wherein the increment in which the dose of memantine is increased in successive dosages is less than 5 mg of memantine.

18. The method of claim 17 wherein the dose of memantine is increased by an increment of about 0.25 to about 0.5 mg each day until the maintenance dose is reached.
